(19) United States
(12) Patent Application Publication
(10) Pub. No.: US 2015/0114395 A1
(43) Pub. Date: Apr. 30, 2015

(54) METHOD AND ARRANGEMENT FOR DETERMINING A VENTILATION NEED SPECIFIC FOR A PATIENT

(71) Applicant: GENERAL ELECTRIC COMPANY, Schenectady, NY (US)

(72) Inventors: Erkki Heinonen, Helsinki (FI); Tom Haggblom, Vantaa (FI)

(73) Assignee: GENERAL ELECTRIC COMPANY, Schenectady, NY (US)

(21) Appl. No.: 14/065,918

(22) Filed: Oct. 29, 2013

(51) Int. Cl. A61M 16/00; A61B 5/085; A61M 16/12; A61M 16/08; A61B 5/091; A61B 5/083; A61B 5/08; A61B 5/00

(52) U.S. Cl. CPC A61M 16/0069; A61M 16/0003; A61B 5/0816; A61B 5/085; A61B 5/4836; A61M 16/0051; A61M 16/0883; A61B 5/091; A61B 5/0836; A61M 16/12; A61M 2016/0027; A61M 2230/46
USPC 128/204.23

(57) ABSTRACT

A method for determining a ventilation need specific for a patient is disclosed herein. The method includes providing a breath gas with a machine ventilator circuit from a starting pressure to lungs to start inspiration, and filling lungs to a predetermined breath gas pressure level. The method also includes determining in a control unit a filling volume of the breath gas needed to achieve the predetermined breath gas pressure level from the starting pressure, and determining in the control unit a lung elastic property based on a relationship between the determined filling volume of the breath gas and differences in the starting pressure and the predetermined breath gas pressure level. The method also includes determining in the control unit a respiration rate exploiting at least the lung elastic property. A corresponding arrangement is also provided.

### BACKGROUND OF THE INVENTION

This disclosure relates generally to a method and arrangement for determining a ventilation need specific for a patient.

Ventilation provides oxygen in breathing gas to patient lungs during inspiration and clearance of carbon dioxide (CO₂) mixed with expiration gas. The rate of oxygen consumption and CO2 production correlate closely and depends on the body metabolism.

During intensive care and anesthesia the subject may be unable to maintain ventilation to meet the metabolic demand and mechanical ventilation is used to support or replace the subject's spontaneous breathing.

Clinician controls ventilation rate to maintain appropriate subject's CO2 on physiological level. Measured end-expiratory CO2 (EtCO2) concentration is used as indicator of the CO2 level. Typical EtCO2 value is around 5% but on certain circumstances the optimum value may deviate from this.

Metabolism and CO2 production varies between subjects. This depends e.g. on subject size, age, gender, anxiety level, etc. The anxiety varies during the mechanical ventilation and also treatment actions vary the required CO2 clearance. To maintain the optimal subject CO2 level the ventilation rate must be tuned.

Ventilation rate can be regulated automatically to maintain the given target patient CO2 level exploiting the measured EtCO2 value to control ventilation rate to match the measured value with given target. Problem in such ventilation automation is to identify initial ventilation settings. User given subject information has been utilized for this, which poses safety risk of erroneous values. Also test breaths with settings safe for any patient to measure subject airway volume, i.e. the anatomical dead space, and using correlations from this to patient weight, and further to metabolism end ventilation settings characterize subject lung characteristics has been used. Problem with this kind of determination is that anatomic dead space measurement requires flow sensor at the subject connection to ventilation breathing system, and such measurement is not included in anesthesia standard. Anesthesia standard using the anesthesia ventilator embedded flow sensor cannot be applied for this purpose since that requires precise time synchronization with the gas concentration signal at patient connection. That is only possible when the sensors are located close to each other, or at least the time difference between the signals is well defined. This is not true when anesthesia ventilator sensors measure the flow through resistive and large-volume anesthesia breathing system.

Tidal volume (Vt) and respiration rate (RR) define the ventilation rate. RR still divides to inspiration (ti) and expiration (te) times. These parameters are highly specific to subject characteristics. Vt may vary between 50 mL and 700 mL, and even beyond. RR typically ranges from 8 to 25, and as well even beyond. With this large variation initial setting compatible with subject characteristics already from the first breath is important to the ventilation safety.

### BRIEF DESCRIPTION OF THE INVENTION

The above-mentioned shortcomings, disadvantages and problems are addressed herein which will be understood by reading and understanding the following specification.

In an embodiment, a method for determining a ventilation need specific for a patient includes providing a breath gas with a machine ventilator circuit from a starting pressure to lungs of the patient to start inspiration, and filling lungs to a predetermined breath gas pressure level. The method also includes determining in a control unit a filling volume of the breath gas needed to achieve the predetermined breath gas pressure level from the starting pressure, and determining in the control unit a lung elastic property based on a relationship between the determined filling volume of the breath gas and differences in the starting pressure and the predetermined breath gas pressure level. The method also includes determining in the control unit a respiration rate exploiting at least the lung elastic property.

In another embodiment, an arrangement for determining a ventilation need specific for a patient includes a machine ventilator circuit configured to connect to lungs of the patient and which machine ventilator circuit comprises an inspiration delivery unit for delivering a gas flow to assist an inspiration, at least one flow sensor (32, 35) for measuring said gas flow and an expiration circuit for controlling a discharge of an expiration gas. The arrangement also includes a control unit configured to control an operation of the machine ventilator circuit. The machine ventilator circuit is configured to provide a breath gas from a starting pressure to lungs of the patient to start inspiration, and to fill lung to a predetermined breath gas pressure level. The control unit is configured to determine a filling volume of the breath gas, based on the measured gas flow, needed to achieve the predetermined breath gas pressure level from the starting pressure, and to determine a lung elastic property based on a relationship between the determined filling volume of the breath gas and differences in the starting pressure and the predetermined breath gas pressure level. The control unit is also configured to determine a respiration rate exploiting at least the lung elastic property.

In yet another embodiment, a method for determining a ventilation need specific for a patient includes providing a breath gas with a machine ventilator circuit from a starting pressure to lungs of the patient to start inspiration, and filling lungs to a predetermined breath gas pressure level. The method also includes determining in a control unit a filling volume of the breath gas needed to achieve the predetermined breath gas pressure level from the starting pressure, and determining in the control unit a lung elastic property based on a relationship between the determined filling volume of the breath gas and differences in the starting pressure and the predetermined breath gas pressure level. The method also includes determining in the control unit a target breath volume, which is based on one of the determined filling volume of the breath gas and some other relationship to the lung elastic property, and determining in the control unit a respiration rate exploiting the lung elastic property and the target breath volume. The method also includes releasing in an expiration circuit the pressure of lungs from the predetermined breath gas pressure level, and determining in the control unit a time needed for the release of the pressure of the lungs. The method also includes receiving in the control unit an inspiration to expiration time ratio, and determining in the control unit an expiration time based on the inspiration to expiration time ratio, the time needed for the release of the pressure of the lungs, and the respiration rate. The method also includes determining in the control unit an inspiration time based on the determined expiration time and the determined respiration rate.

Various other features, objects, and advantages of the invention will be made apparent to those skilled in art from the accompanying drawings and detailed description thereof.

### BRIEF DESCRIPTION OF THE DRAWINGS

FIG. 1 illustrates an operational diagram of an arrangement for determining a ventilation need specific for a patient;

FIG. 2 is an operational diagram of an arrangement for determining a ventilation need specific for a patient according to another embodiment employed in anesthesia;

FIG. 3 presents the breathing circuit pressure, flow and volume of the test breath;

FIG. 4 presents a general method for determining a ventilation need; and

FIG. 5 presents a detailed method for determining the ventilation need of FIG. 4.

### DETAILED DESCRIPTION OF THE INVENTION

Specific embodiments are explained in the following detailed description making a reference to accompanying drawings. These detailed embodiments can naturally be modified and should not limit the scope of the invention as set forth in the claims.

The embodiments are directed to an arrangement and a method which may be useful in connection of mechanical ventilation therapy typically during intensive care or anesthesia. More particularly the method may be useful in connection of target controlled ventilation where the method can be applied to determine patient specific ventilation need, such as initial ventilation settings.

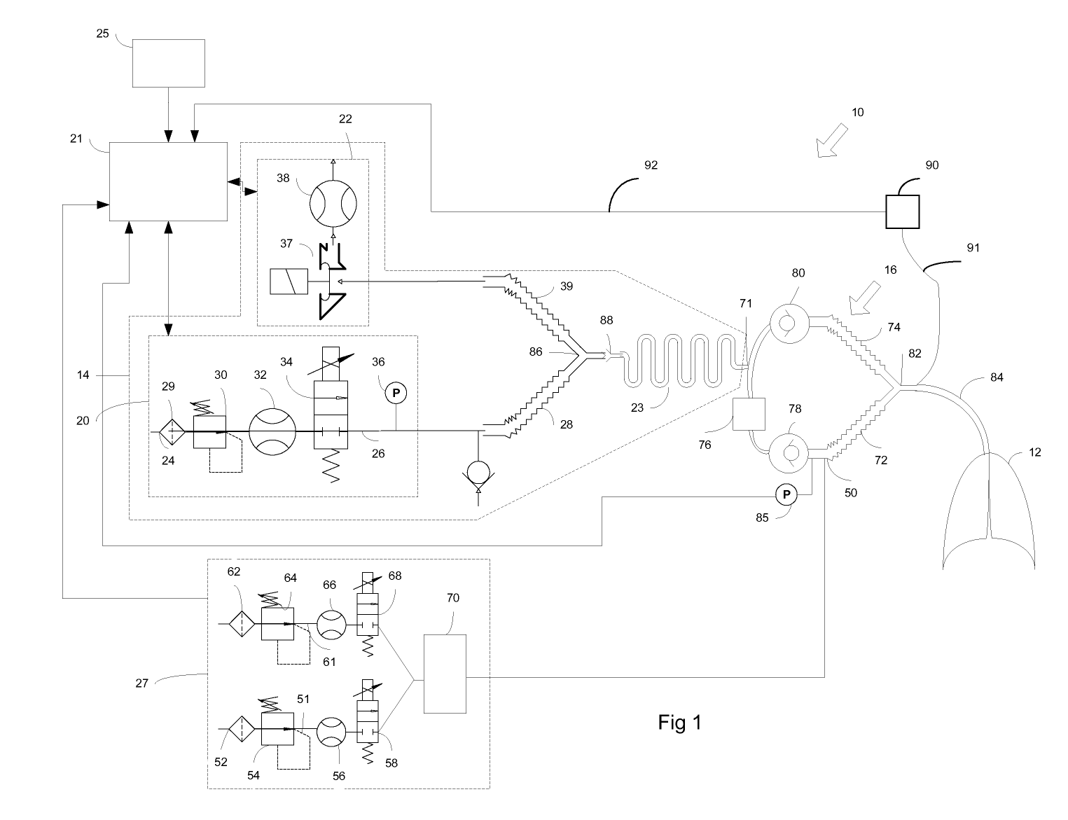

The arrangement 10 for providing an inspiration gas to lungs 12 of a patient utilizing a re-breathing system is shown in FIG. 1. The arrangement 10 comprises a machine ventilator circuit 14 for assisting breathing functions of the patient and to exchange the gas in the lungs, a breathing circuit 16 for connecting lungs of the patient, and a control unit 21 for controlling an operation of the machine ventilator circuit or even the whole arrangement 10. The arrangement 10 shown in FIG. 1 may also comprise a user interface 25 for entering any information needed while ventilating the subject and a gas mixer 27 for supplying a fresh gas for the subject breathing.

The machine ventilator circuit 14 generally comprises an inspiration delivery unit 20 for delivering the gas such as drive gas needed to enable an inspiration of the subject, an expiration circuit 22 for controlling a discharge of the expiration gas and a reciprocating unit 23 such as a well-known bellows and bottle combination, where the bellows are arranged within the bottle, or a long gas flow channel as shown in FIG. 1 for compressing the gas under a control of the drive gas pressure towards lungs of the subject to facilitate the inspiration. Both the inspiration delivery unit 20 and the expiration circuit 22 are controlled by the control unit 21.

As illustrated in FIG. 1, the inspiration delivery unit 20 comprises a compressed gas interface 24 connected to a compressed gas supply (not shown). The compressed gas can be either oxygen or air. Also a mechanism selecting the other if one gets de-pressurized can be applied (not shown). The inspiration delivery unit 20 comprises also a filter 29 for filtering impurities, a pressure regulator 30 for regulating a pressure of gases flowing from the gas interface, a flow sensor 32 for measuring an inspiration delivery flow from the gas interface and a flow control valve 34 for opening or closing the inspiration delivery flow. The flow sensor 32 and flow control valve 34 are each coupled to the control unit 21 to control the inspiration delivery to the subject lungs 12. Further the inspiration delivery unit 20 may comprise a pressure sensor 36 for measuring the gas pressure flowing along the conduit 26 and an inspiration branch 28 towards the reciprocating unit 23.

The expiration circuit 22 comprises an expiration valve 37 for discharging the expiration gas and a flow sensor 38, which is optional, for measuring the flow discharged through the expiration valve 37. The expiration circuit is in flow connection along an expiration branch 39 with the reciprocating unit 23.

The gas mixer 27 is arranged to supply the fresh gas through a fresh gas outlet 50 to the breathing circuit 16 for the subject breathing. Typically the fresh gas comprises of oxygen and air or nitrous oxide. Oxygen is delivered through an oxygen delivery line 51 comprising of a filter 52, a pressure regulator 54, an oxygen flow sensor 56 and an oxygen flow control valve 58. The air is delivered through an air delivery line 61 comprising of filter 62, a pressure regulator 64, an air flow sensor 66, and air flow control valve 68. For a delivery of nitrous oxide respective components may be provided (not shown). After metering the individual gas flows, they are merged together for fresh gas mixture delivered to a vaporizer 70 which completes the fresh gas mixture with a volatile anesthesia agent vapor before delivery to the breathing circuit 16 at the fresh gas outlet 50 and to the subject breathing.

The breathing circuit 16, which is operably connected to the machine ventilator circuit 14 at a breathing circuit connection 71 and to the fresh gas outlet 50, comprises an inspiration limb 72 for an inspired gas, an expiration limb 74 for an exhaled gas, a carbon dioxide (CO2) remover 76 such as CO2 absorber to remove or absorb carbon dioxide from the exhaled gas coming from the subject lungs 12, a first one-way valve 78 for an inspired gas to allow an inspiration through the inspiration limb 72, a second one-way valve 80 for an expired gas to allow an expiration through the expiration limb 74, a branching unit 82 such as a Y-piece having at least three limbs, one of them being for the inspired gas, a second one being for the expired gas and a third one being for both the inspired and expired gases and being connectable by means of the patient limb 84 to the lungs 12 of the subject. Thus the patient limb may provide both the inspiration gas to the lungs and expiration gas from the lungs. The patient limb may be between the branching unit 82 and the lungs 12 of the subject. Also the breathing circuit may comprise a pressure sensor 85 for measuring a pressure of the breathing circuit 16.

During the inspiration phase of the machine ventilation the expiration circuit 22 of the machine ventilator circuit 14 closes the expiration valve 37 under the control of the control unit 21. This guides the inspiration gas flow from the inspiration delivery unit 20 through the inspiration branch 28 of a gas branching connector 86 and through the connection 88 of the reciprocating unit 23 pushing the breathing gas out from the breathing circuit connection 71 to the breathing circuit 16. The inspiration gas delivery unit 20 controlled by the control unit 21 delivers the gas flow either to reach the given gas volume or a pressure at subject lungs. For this control at least one of the flow sensors 32, 56, 66 for measuring the inspiration flow and the pressure sensor 85 of the breathing circuit 16 may be exploited in the embodiment of FIG. 1.

At the end of the inspiration phase the breathing circuit 16 and the subject lungs are pressurized. For the expiration under the control of the control unit 21 the inspiration delivery flow control valve 34 is closed stopping the inspiration delivery and the expiration valve 37 is opened to allow the gas release from the expiration branch 39 of the drive gas branching connector 86 and further through the connection 88 from the reciprocating unit 23. This allows the pressure release and breathing gas flow from breathing circuit 16 and the lungs 12 of the subject to the reciprocating unit 23. The breathing gas flows from the subject 12 through the patient limb 84, the branching unit 82, the expiration limb 74, the second one-way valve 80 for the expired gas and the breathing circuit connection 71 to the reciprocating unit 23. The pressure release is controlled for a desired expiration pressure such as a positive end expiration pressure (PEEP) target, which may be set exploiting the user interface 25. For this control the control unit 21 may exploit the breathing circuit pressure measured by the pressure sensor 85 and the expiration valve 37. The expiration gas flow may be measured exploiting the flow sensor 38 located at the outlet the expiration valve 37 as shown in FIG. 1 or at any location on the expiration pathway from patient limb 84 to the expiration valve 37.

FIG. 1 presents also a gas analyzer 90 to measure subject breathing gas concentrations. Such analyzer can be either sidestream type that suctions a sample gas stream through sampling line 91 for analysis or mainstream type where the analysis occurs in the gas stream in the patient limb 84. The analyzer communicates gas concentrations to control unit 21 through communication line 92. Gas analyzer can be of any known type able to measure particular gas concentration. For CO2 infrared absorption is the most commonly used measurement principle.

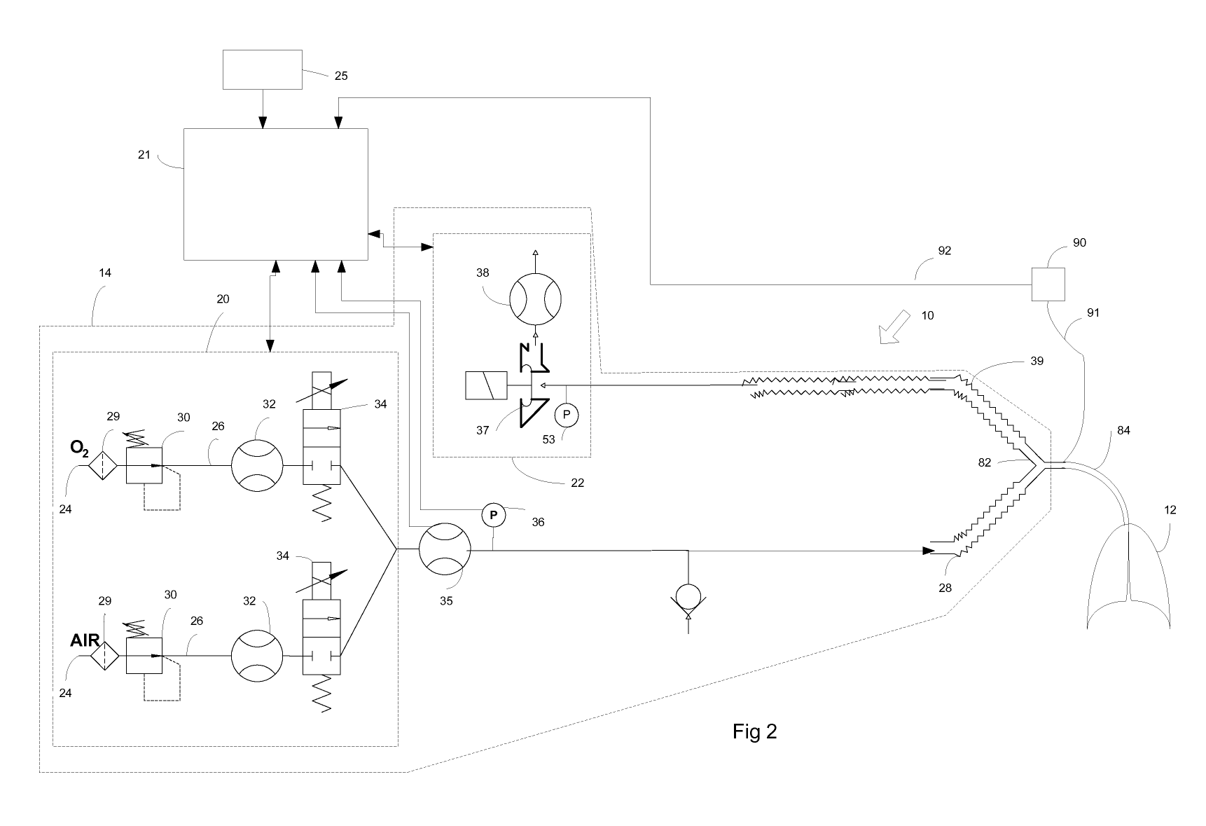

FIG. 2 shows the arrangement 10 of another embodiment having an open breathing system. Such system neither has separate fresh gas supply nor dedicated drive gas but the drive gas is the mixture of oxygen and air provided directly through its inspiration branch 28, the branching unit 82 and the patient limb 84 to lungs 12 of the subject. In this setting the inspiration delivery unit 20 of the machine ventilator circuit 14 comprises two separate conduits 26 for the gas such as the drive gas. One of those conduits may be for oxygen and another one may be for the air. Both conduits 26 comprises the compressed gas interfaces 24 for inspiration delivery connected to compressed gas supplies (not shown), the filter 29, the pressure regulators 30, the flow sensors 32 for measuring the inspiration delivery flow and the flow control valves 34. These components have been introduced hereinbefore when explaining the FIG. 1 embodiment. After metering the individual gas flows to produce the required gas mixture having the desired O₂ concentration and desired total flow rate the gas flows are merged to a gas mixture, which may still be measured for cross referencing the sensor operational condition with total flow sensor 35. Also it is desired to measure the pressure of the merged gas mixture by means of the pressure sensor 36.

In FIG. 2 the expiration circuit 22 of the open breathing system just as the FIG. 1 embodiment also comprises the expiration valve 37 and optionally the flow sensor 38 connected either downstream or upstream to the expiration valve 37. Further in this embodiment the expiration circuit 22 may comprise a pressure sensor 53 for measuring the pressure prevailing in the expiration branch 39. Gas analysis occurs similarly to FIG. 1.

In automatic ventilation control the control unit 21 may exploit the measured exhaled CO2 concentration and compare the value with target value given through user interface 25. If the measured value is higher than the target, control unit 21 increases the subject lung ventilation either by instructing larger inspiration volumes or more frequent volumes. Respectively, if the measured value is lower than the target, the ventilator control reduces the ventilation. If the values match the ventilation is maintained unchanged.

Safe ventilation needs, such as ventilation settings, vary a lot between subjects and are closely related to subject size. This ventilation need distributes to the breath volume and respiration rate. Whereas safe breath volume for one patient may be 700 mL, for another patient 100 mL may be too much. Appropriate respiration rate is defined to match the ventilation with the need. To begin mechanical ventilation, safe subject specific ventilation needs and its optimal distribution to components is therefore important. To determine these, particular breath, such as a test breath, is useful. Particular importance this determination is to initiate the automatic ventilation control.

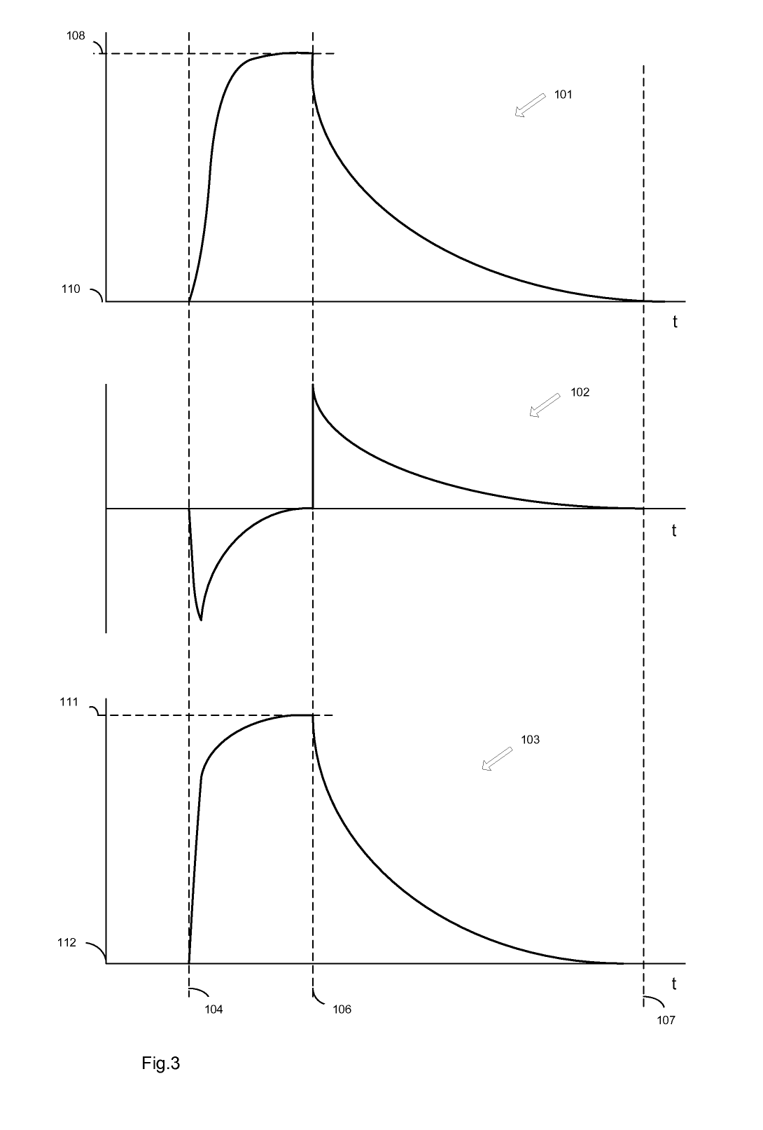

The breath according to an embodiment can pressurize the lungs to a pressure level safe to any connected patient. Such pressure is e.g. 10-15 cmH2O. FIG. 3 presents the breath pressure 101, flow 102 and volume 103 values on ordinate as a function of time on abscissa. Dotted line 104 designates time for beginning of inspiration period, 106 end of inspiration and begin of expiration, and 107 end of expiration flow. Horizontal line 108 illustrates predetermined breath gas pressure level, such as a target pressure of the test breath, which may be either system default or user given through the user interface 25. The gas volume needed for pressurization is determined as integral of the inspiration flow between the beginning of inspiration period 104 and the end of inspiration filling period 106. Inspiration filling pressure, if desired, can be measured at the end of inspiration 106 at condition where no gas is flowing to or from subject lungs. At this moment the measured pressure equals the subject lung pressure. The predetermined breath gas pressure level may deviate in some degree from the measured value, but the difference is relatively insignificant and therefore in this description the predetermined breath gas pressure level also covers the measured value at the end of inspiration. Lung elastic property may now be calculated exploiting the differences in the predetermined breath gas pressure level 108 and starting pressure 110 and the respective filling volume 111, such as a tidal volume.

The test breath gives information also about patient airway status. The expiration time between end of inspiration and begin of expiration 106 and end of expiration flow 107 measures minimum expiration time to allow lung emptying. Knowing this is important since patients with obstructive airways develop spontaneous static pressure in the lungs if expiration is incomplete. This may damage the lung tissue and also overload patient heart with the static circulatory pressure load. This minimum expiration time can then be useful to control the breath cycle in order to provide sufficient expiration.

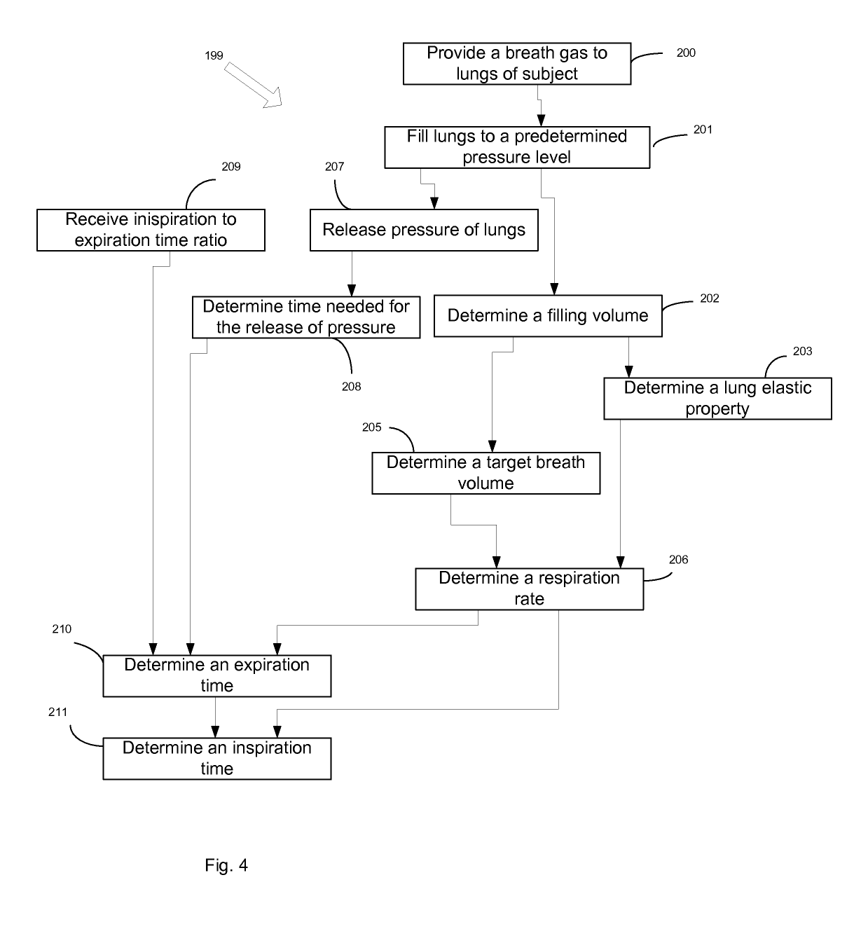

FIG. 4 depicts a method 199 for determining the ventilation need specific for the patient. At step 200 the breath gas is provided to lungs of the patient. At step 201 lungs are filled to a predetermined breath gas pressure level 108. This pressure may be a pre-programmed default value or the user can have a value according to his own preference for the particular patient ventilation. The machine ventilator circuit 14 may pressurize the arrangement 10 and the lungs 12 of the patient connected to it up to this predetermined pressure level. As explained hereinbefore this pressure can be measured (this step not shown in FIG. 4) e.g. with the pressure sensor 36, 53 or 85 connected to measure breathing circuit pressure shown in FIG. 1 or 2, which it is not quite necessary, but if however measured this measured value can replace the predetermined breath gas pressure level, which measured value can also be considered as the predetermined breath gas pressure level, because the deviation between these values is not so significant. This means that the step 201 may cover the predetermined pressure level measurement, if such measurement is made.

The filling volume 111 to pressurize the lungs to achieve the predetermined breath gas pressure level 108 from the starting pressure 110 is determined at step 202. The determination can be made in the control unit 21 by exploiting the measured value of the filling volume. At step 203 the lung elastic property may be determined in the control unit 21 based on a relationship between the determined filling volume of the breath gas and differences in the starting pressure 110 and the predetermined breath gas pressure level 108. This calculation is a ratio of these two and may be called as compliance C when the determined filling volume is divided with the difference between the starting pressure and the predetermined breath gas pressure or may be called as elastance when pressure difference, which is the difference between the starting pressure and the predetermined breath gas pressure, is divided with the determined filling volume. The ventilation need is determined at step 204 in the control unit 21 exploiting the determined lung elastic property.

According to step 205 a target breath volume is determined in the control unit 21. It can be based on, typically equal to, the determined filling volume 111 directly or utilizing some other relationship to the lung elastic property. A respiration rate is determined at step 206 in the control unit 21 exploiting the determined lung elastic property and the determined target breath volume. The ventilation need may be expressed as a product of respiration rate (RR) and the filling volume.

At step 207 the pressure is released from lungs 12 in an expiration circuit 22 to allow expiration under the control of the control unit 21. The pressure is released from the predetermined breath gas pressure level 108 typically back to the starting pressure 110. The time needed for the release is determined at step 208 in the control unit 21. This can be called minimum expiration time. At step 209 an inspiration to expiration time ratio is received by the control unit 21. Typically this is given through the user interface 25 by the user. Based on this inspiration to expiration time ratio, the minimum expiration time needed for the release of the pressure of the lungs, and the determined respiration rate, an expiration time can be determined according to step 210. Further an inspiration time can be determined at step 211 based on the determined expiration time and the respiration rate. The inspiration time determination may be made by subtracting the determined expiration time from breath time determined as 60/respiration rate.

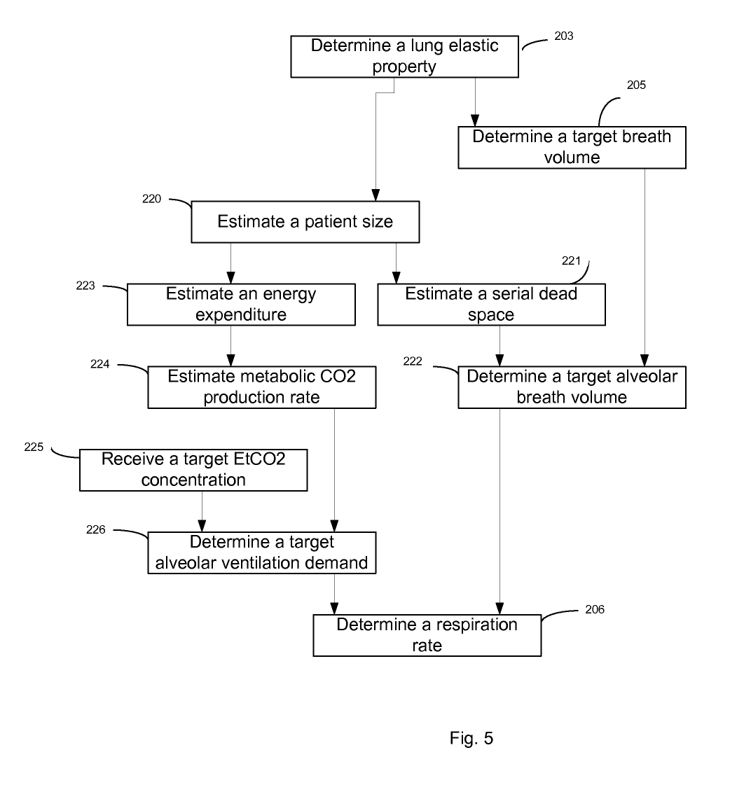

FIG. 5 depicts a detailed example of procedure to determine respiration rate from lung elastic property, step 203, on FIG. 4. Steps 203, 205 and 206 of FIG. 5 equals the respective steps on FIG. 4.

At step 220 the subject size is estimated from the determined lung elastic property, because lung elastic property expresses good correlation to subject size. Subject size can be subject weight, height, or body surface area. Such correlations are available on medical literature.

As well medical literature provides correlation between subject anatomic dead space, which is the gaseous volume of subject airways, and the estimated subject size. This anatomical dead space provides a breathing gas pathway to and from the lung where the breathing gas interfaces blood circulation for gas exchange. Thus, at the beginning of inspiration this anatomical dead space is filled with gas from previous expiration, which will be inspired at first back to lungs before new fresh breathing gas. Because this reverting volume is already equilibrated with the lung concentrations, that does not enhance any more the alveolar gas exchange. Furthermore, at the end of inspiration this anatomical dead space is filled with fresh inspiration gas, which becomes expired first at the beginning of expiration without participating the alveolar gas exchange. Exploiting this correlation a serial dead space, depicting the gaseous volume of subject airways, which is the anatomical dead space, and the patient limb 84, is estimated on step 221. The patient limb also includes expired gas at the beginning of inspiration, because both the expired and inspired gas flow though this tube. Thus this serial dead space is the breath gas volume with insufficient subject gas exchange on the alveoli of the lungs or in other words it is a part of the breath volume that does not participate subject gas exchange on the alveoli of the lungs. A target alveolar breath volume is determined on step 222 as a difference of the determined target breath volume of step 205 and the estimated serial dead space.

Scientific studies have reported correlation between energy expenditure and patient size. Exploiting this correlation energy expenditure is estimated on step 223. Relationship between metabolic CO2 production rate and the estimated energy expenditure is also well known and exploiting this relationship the metabolic carbon dioxide (CO2) production rate is estimated on step 224.

On step 226 the target alveolar ventilation demand is determined as ratio of the estimated metabolic CO2 production rate and a target end tidal CO2 concentration received on step 225. Thus target alveolar ventilation demand being determined by the metabolism correlates with the lung elastic property and can be determined from the lung elastic property. The alveolar ventilation demand may be expressed as a product of respiration rate (RR) and the alveolar breath volume. The target end tidal CO2 concentration may be given through the user interface 25 by the user. Dividing the determined target alveolar ventilation demand with the determined target alveolar breath volume gives the respiration rate. Thus, the procedures of FIG. 4 and FIG. 5 establish the set of initial subject specific ventilation parameters to begin subject ventilation by a test breath measuring lung elastic property and minimum required lung emptying time. This set of initial parameters may comprise the filling volume, inspiration time and expiration time. The filling volume can be useful either directly if the machine ventilator circuit 14 is programmed to deliver the gas in volume control mode. Alternatively, the machine ventilator circuit 14 may be programmed to reach and maintain constant pressure during inspiration phase. In such pressure controlled ventilation mode the filling volume needed for pressurization is measured. After the breath the determined filling volume can be compared with the target breath volume and modify the ventilation pressure for the next breath in order to match the determined filling volume with the target breath volume.

As can be understood hereinbefore it is advantageous according to some embodiments described that the initial ventilation need adapted to subject characteristics may be determined with a test breath. On this breath lung is pressurized to a pressure level safe for any patient, e.g. 10 cmH2O and the gas volume (dV) required for this is measured for example exploiting flow sensors 32, 56, 66. The relationship between this volume and the pressure change designates for the elastic properties of the patient lungs. The lung elastic properties also correlate with physiological patient characteristics. This lung elastic property determines optimal ventilation pattern. Because the elastic property may change on patient lung illnesses, knowing this gives a basis to optimize the ventilation superior to patient demographic information.

Further as explained hereinbefore the most widely known elastic lung characteristic is compliance, which is calculated as C=dV/dP. Correlation with the C to various physiological patient characteristics is widely published. These already known correlations can advantageously be utilized in some embodiments discussed hereinbefore.

Another characteristic defining the patient specific ventilation need is the flow resistance. The resistance tends to increase especially on obstructive lung diseases. Important for the patient specific ventilation need is that the expiration time is long enough to allow lung emptying before next inspiration. Would this not occur patient lungs remain distended due to remaining gas volume. This may be for benefit for the gas exchange if controlled correctly, but also harmful to the patient if left unnoticed.

The embodiments disclosed herein thus may provide the patient specific initial ventilation values. These may not provide the expected target EtCO2 concentration but instead may provide safe begin for ventilation. Especially in various sicknesses deviations from these initial values may be needed. Therefore the embodiments may be useful for instance to determine the initial settings for ventilation feedback to automatically adjust ventilation rate to match the measured EtCO2 value with the clinician given target.

The embodiments may enable new fully automatic ventilation control without relying any given unverified background information like patient demographics. The method also considers patient airway status to allow sufficient expiration time for lung emptying.

The written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for determining a ventilation need specific for a patient, said method comprising:
providing a breath gas with a machine ventilator circuit from a starting pressure to lungs of the patient to start inspiration;
filling lungs to a predetermined breath gas pressure level;
determining in a control unit a filling volume of the breath gas needed to achieve said predetermined breath gas pressure level from said starting pressure;
determining in said control unit a lung elastic property based on a relationship between said determined filling volume of the breath gas and differences in said starting pressure and said predetermined breath gas pressure level; and
determining in said control unit a respiration rate exploiting at least said lung elastic property.

2. The method according to claim 1, further comprising determining in said control unit a target breath volume, which is based on one of said determined filling volume of the breath gas and some other relationship to said lung elastic property; and when determining said respiration rate exploiting besides said lung elastic property but also said target breath volume.

3. The method according to claim 2, further comprising releasing in an expiration circuit the pressure of lungs from said predetermined breath gas pressure level; and determining in said control unit a time needed for the release of the pressure of the lungs.

4. The method according to claim 3, further comprising receiving in said control unit an inspiration to expiration time ratio; and determining in said control unit an expiration time based on said inspiration to expiration time ratio, said time needed for the release of the pressure of the lungs, and said respiration rate.

5. The method according to claim 4, further comprising determining in said control unit an inspiration time based on said determined expiration time and said determined respiration rate.

6. The method according to claim 1, wherein said lung elastic property is a compliance, which is a ratio of said determined filling volume of the breath gas to differences in said starting pressure and said predetermined breath gas pressure level.

7. The method according to claim 1, further comprising estimating in said control unit a patient size based on said determined lung elastic property; estimating in said control unit a serial dead space based on said estimated patient size, which serial dead space is the breath gas volume with insufficient subject gas exchange on the alveoli of the lungs; and determining in said control unit based on said estimated serial dead space a target alveolar breath volume.

8. The method according to claim 7, wherein said determined target alveolar breath volume is a difference of determined target breath volume, which is one of equal to said determined filling volume of the breath gas and based on said lung elastic property, and said estimated serial dead space.

9. The method according to claim 7, further comprising estimating in said control unit an energy expenditure of the patient based on said estimated patient size; estimating in said control unit metabolic carbon dioxide production rate based on said estimated energy expenditure; and receiving in said control unit a target end tidal carbon dioxide concentration.

10. The method according to claim 7, further comprising determining in said control unit a target alveolar ventilation demand based on said estimated metabolic carbon dioxide production rate and said received target end tidal carbon dioxide concentration.

11. The method according to claim 10, wherein said determined target alveolar ventilation demand is a ratio of said estimated metabolic carbon dioxide production rate and said received target end tidal carbon dioxide concentration.

12. The method according to claim 10, further comprising determining in said control unit the respiration rate by dividing said determined target alveolar ventilation demand with said determined target alveolar breath volume.

13. An arrangement for determining a ventilation need specific for a patient, said arrangement comprising:
a machine ventilator circuit configured to connect to lungs of the patient and which machine ventilator circuit comprises an inspiration delivery unit for delivering a gas flow to assist an inspiration, at least one flow sensor for measuring said gas flow and an expiration circuit for controlling a discharge of an expiration gas; and
a control unit configured to control an operation of said machine ventilator circuit,
wherein said machine ventilator circuit is configured to provide a breath gas from a starting pressure to lungs of the patient to start inspiration; and to fill lungs to a predetermined breath gas pressure level; and
wherein said control unit is configured to determine a filling volume of the breath gas, based on said measured gas flow, needed to achieve said predetermined breath gas pressure level from said starting pressure; to determine a lung elastic property based on a relationship between said determined filling volume of the breath gas and differences in said starting pressure and said predetermined breath gas pressure level; and to determine a respiration rate exploiting at least said lung elastic property.

14. The arrangement according to claim 13, further comprising a gas mixer configured to supply a fresh gas for the subject breathing; and a breathing circuit configured to connect lungs of the subject with said machine ventilator circuit and said gas mixer to provide an inspiration gas including the fresh gas for the subject breathing, said breathing circuit comprising a branching unit having at least three limbs, one of them being for an inspired gas, a second one being for an expired gas and a third one being for both the inspired and expired gases.

15. The arrangement according to claim 13, wherein said control unit is further configured to determine a target breath volume, which is based on one of said determined filling volume of the breath gas and some other relationship to said lung elastic property; and when determining said respiration rate exploiting besides said lung elastic property but also said target breath volume.

16. The arrangement according to claim 15, wherein said expiration circuit of said machine ventilator circuit is further configured to release the pressure of lungs from said predetermined breath gas pressure level; and wherein said control unit is configured to determine a time need for the release of the pressure of the lungs.

17. The arrangement according to claim 16, wherein said control unit is configured to receive an inspiration to expiration time ratio; and to determine an expiration time based on said inspiration to expiration time ratio, said time needed for the release of the pressure of the lungs, and said respiration rate.

18. The arrangement according to claim 17, wherein said control unit is configured to determine an inspiration time based on said determined expiration time and said determined respiration rate.

19. A method for determining a ventilation need specific for a patient, said method comprising:
providing a breath gas with a machine ventilator circuit from a starting pressure to lungs of the patient to start inspiration;
filling lungs to a predetermined breath gas pressure level;
determining in a control unit a filling volume of the breath gas needed to achieve said predetermined breath gas pressure level from said starting pressure;
determining in said control unit a lung elastic property based on a relationship between said determined filling volume of the breath gas and differences in said starting pressure and said predetermined breath gas pressure level;
determining in said control unit a target breath volume, which is based on one of said determined filling volume of the breath gas and some other relationship to said lung elastic property;
determining in said control unit a respiration rate exploiting said lung elastic property and said target breath volume;
releasing in an expiration circuit the pressure of lungs from said predetermined breath gas pressure level;
determining in said control unit a time needed for the release of the pressure of the lungs;
receiving in said control unit an inspiration to expiration time ratio;
and determining in said control unit an expiration time based on said inspiration to expiration time ratio, said time needed for the release of the pressure of the lungs, and said respiration rate; and
determining in said control unit an inspiration time based on said determined expiration time and said determined respiration rate.

20. The method according to claim 19, further comprising estimating in said control unit a patient size based on said determined lung elastic property;
estimating in said control unit a serial dead space based on said estimated patient size;
determining in said control unit based on said estimated serial dead space a target alveolar breath volume:
estimating in said control unit an energy expenditure of the patient based on said estimated patient size;
estimating in said control unit metabolic carbon dioxide production rate based on said estimated energy expenditure;
receiving in said control unit a target end tidal carbon dioxide concentration;
determining in said control unit a target alveolar ventilation demand based on said estimated metabolic carbon dioxide production rate and said received target end tidal carbon dioxide concentration; and
determining in said control unit the respiration rate by dividing said determined target alveolar ventilation demand with said determined target alveolar breath volume.
